(19) United States
(12) Patent Application Publication
(10) Pub. No.: US 2008/0119482 A1
(43) Pub. Date: May 22, 2008

(54) Method for the treatment of attention deficit hyperactivity disorder

(76) Inventor: Mikael Goeran Dolsten, Scarsdale, NY (US)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(21) Appl. No.: 11/940,655

(22) Filed: Nov. 15, 2007

Related U.S. Application Data
(63) Continuation of application No. 11/218,107, filed on Sep. 1, 2005, now abandoned.
(60) Provisional application No. 60/606,938, filed on Sep. 3, 2004.

(51) Int. Cl. A61K 31/496

(52) U.S. Cl. 514/254.06

(57) ABSTRACT

The invention relates to a method for the treatment of Attention Deficit Hyperactivity Disorder comprising the administration of a therapeutically effective amount of flibanserin.

This application is a continuation of U.S. application Ser. No. 11/218,107, filed on Sep. 1, 2005, which claims priority benefit, as does the present application, to U.S. Provisional Application Ser. No. 60/606,938, filed on Sep. 3, 2004.

The invention relates to a method for the treatment of Attention Deficit Hyperactivity Disorder (ADHD) comprising the administration of a therapeutically effective amount of flibanserin.

### DESCRIPTION OF THE INVENTION

The compound 1-[2-(4-(3-trifluoromethyl-phenyl)piperazin-1-yl)ethyl]-2,3-dihydro-1H-benzimidazol-2-one (flibanserin) is disclosed in form of its hydrochloride in European Patent Application EP-A-526434 and has the following chemical structure:

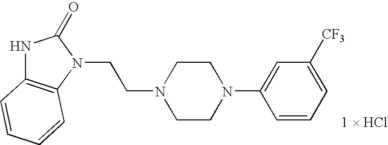

Flibanserin shows affinity for the 5-HT_{1A} and 5-HT₂-receptor. It is therefore a promising therapeutic agent for the treatment of a variety of diseases, for instance depression, schizophrenia, and anxiety.

The instant invention relates to a method for the treatment of Attention Deficit Hyperactivity Disorder (ADHD) comprising the administration of a therapeutically effective amount of flibanserin, optionally in form of the pharmacologically acceptable acid addition salts thereof.

Another embodiment of the invention relates to the use of flibanserin, optionally in form of the pharmacologically acceptable acid addition salts thereof for the preparation of a medicament for the treatment of Attention Deficit Hyperactivity Disorder (ADHD).

Attention Deficit Hyperactivity Disorder (ADHD) is a disorder which may be divided into three subtypes, according to the main features associated with the disorder: inattentiveness, impulsivity, and hyperactivity. The three subtypes are ADHD predominantly combined type, ADHD predominantly inattentive type, and ADHD predominantly hyperactive-impulsive type.

Accordingly, in another embodiment the invention is directed to a method for the treatment of Attention Deficit Hyperactivity Disorder (ADHD) of the predominantly combined type comprising the administration of a therapeutically effective amount of flibanserin, optionally in form of the pharmacologically acceptable acid addition salts thereof. Another embodiment of the invention relates to the use of flibanserin, optionally in form of the pharmacologically acceptable acid addition salts thereof for the preparation of a medicament for the treatment of Attention Deficit Hyperactivity Disorder (ADHD) of the predominantly combined type.

In another embodiment the invention is directed to a method for the treatment of Attention Deficit Hyperactivity Disorder (ADHD) of the predominantly inattentive type comprising the administration of a therapeutically effective amount of flibanserin, optionally in form of the pharmacologically acceptable acid addition salts thereof. Another embodiment of the invention relates to the use of flibanserin, optionally in form of the pharmacologically acceptable acid addition salts thereof for the preparation of a medicament for the treatment of Attention Deficit Hyperactivity Disorder (ADHD) of the predominantly inattentive type.

Accordingly, in another embodiment the invention is directed to a method for the treatment of Attention Deficit Hyperactivity Disorder (ADHD) of the predominantly hyperactive-impulsive type comprising the administration of a therapeutically effective amount of flibanserin, optionally in form of the pharmacologically acceptable acid addition salts thereof. Another embodiment of the invention relates to the use of flibanserin, optionally in form of the pharmacologically acceptable acid addition salts thereof for the preparation of a medicament for the treatment of Attention Deficit Hyperactivity Disorder (ADHD) of the predominantly hyperactive-impulsive type.

Flibanserin can optionally used in form of its pharmaceutically acceptable acid addition salts. Suitable acid addition salts include for example those of the acids selected from, succinic acid, hydrobromic acid, acetic acid, fumaric acid, maleic acid, methanesulphonic acid, lactic acid, phosphoric acid, hydrochloric acid, sulphuric acid, tartaric acid and citric acid. Mixtures of the abovementioned acid addition salts may also be used. From the aforementioned acid addition salts the hydrochloride and the hydrobromide, particularly the hydrochloride, are preferred.

Flibanserin, optionally used in form of its pharmaceutically acceptable acid addition salts, may be incorporated into the conventional pharmaceutical preparation in solid, liquid or spray form. The composition may, for example, be presented in a form suitable for oral, rectal, parenteral administration or for nasal inhalation: preferred forms includes for example, capsules, tablets, coated tablets, ampoules, suppositories and nasal spray.

The active ingredient may be incorporated in excipients or carriers conventionally used in pharmaceutical compositions such as, for example, talc, arabic gum, lactose, gelatine, magnesium stearate, corn starch, acqueous or non acqueous vehicles, polyvinyl pyrrolidone, semisynthetic glycerides of fatty acids, benzalconium chloride, sodium phosphate, EDTA, polysorbate 80. The compositions are advantageously formulated in dosage units, each dosage unit being adapted to supply a single dose of the active ingredient. The doses range applicable per day is between 0.1 to 400, preferably between 1.0 to 300, more preferably between 2 to 200 mg.

Each dosage unit may conveniently contain from 0.01 mg to 100 mg, preferably from 0.1 to 50 mg.

Suitable tablets may be obtained, for example, by mixing the active substance(s) with known excipients, for example inert diluents such as calcium carbonate, calcium phosphate or lactose, disintegrants such as corn starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number or layers to achieve delayed release, possibly using the excipients mentioned above for the tablets.

Syrups or elixirs containing the active substances or combinations thereof according to the invention may additionally contain a sweetener such as saccharine, cyclamate, glycerol or sugar and a flavour enhancer, e.g of. a flavouring such as vanilline or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Solutions for injection are prepared in the usual way, e.g of. with the addition of preservatives such as p-hydroxybenzoates, or stabilisers such as alkali metal salts of ethylenediamine tetraacetic acid, and transferred into injection vials or ampoules.

Capsules containing one or more active substances or combinations of active substances may for example be prepared by mixing the active substances with inert carriers such as lactose or sorbitol and packing them into gelatine capsules.

Suitable suppositories may be made for example by mixing with carriers provided for this purpose, such as neutral fats or polyethyleneglycol or the derivatives thereof.

The Examples which follow illustrate the present invention without restricting its scope:

### EXAMPLES OF PHARMACEUTICAL FORMULATIONS

### A) Tablets

| | per tablet | |
|---|---|---|
| flibanserin hydrochloride | 100 | mg |
| lactose | 240 | mg |
| corn starch | 340 | mg |
| polyvinylpyrrolidone | 45 | mg |
| magnesium stearate | 15 | mg |
| | 740 | mg |

The finely ground active substance, lactose and some of the corn starch are mixed together. The mixture is screened, then moistened with a solution of polyvinylpyrrolidone in water, kneaded, wet-granulated and dried. The granules, the remaining corn starch and the magnesium stearate are screened and mixed together. The mixture is compressed to produce tablets of suitable shape and size.

### B) Tablets

| | per tablet | |
|---|---|---|
| flibanserin hydrochloride | 80 | mg |
| corn starch | 190 | mg |
| lactose | 55 | mg |
| microcrystalline cellulose | 35 | mg |
| polyvinylpyrrolidone | 15 | mg |
| sodium-carboxymethyl starch | 23 | mg |
| magnesium stearate | 2 | mg |
| | 400 | mg |

The finely ground active substance, some of the corn starch, lactose, microcrystalline cellulose and polyvinylpyrrolidone are mixed together, the mixture is screened and worked with the remaining corn starch and water to form a granulate which is dried and screened. The sodium-carboxymethyl starch and the magnesium stearate are added and mixed in and the mixture is compressed to form tablets of a suitable size.

### C) Coated Tablets

| | per coated tablet | |
|---|---|---|
| flibanserin hydrochloride | 5 | mg |
| corn starch | 41.5 | mg |
| lactose | 30 | mg |
| polyvinylpyrrolidone | 3 | mg |
| magnesium stearate | 0.5 | mg |
| | 80 | mg |

The active substance, corn starch, lactose and polyvinylpyrrolidone are thoroughly mixed and moistened with water. The moist mass is pushed through a screen with a 1 mm mesh size, dried at about 45° C. and the granules are then passed through the same screen. After the magnesium stearate has been mixed in, convex tablet cores with a diameter of 6 mm are compressed in a tablet-making machine. The tablet cores thus produced are coated in known manner with a covering consisting essentially of sugar and talc. The finished coated tablets are polished with wax.

### D) Capsules

| | per capsule | |
|---|---|---|
| flibanserin hydrochloride | 150 | mg |
| Corn starch | 268.5 | mg |
| Magnesium stearate | 1.5 | mg |
| | 420 | mg |

The substance and corn starch are mixed and moistened with water. The moist mass is screened and dried. The dry granules are screened and mixed with magnesium stearate. The finished mixture is packed into size 1 hard gelatine capsules.

### E) Ampoule Solution

| | | |
|---|---|---|
| flibanserin hydrochloride | 50 | mg |
| sodium chloride | 50 | mg |
| water for inj. | 5 | ml |

The active substance is dissolved in water at its own pH or optionally at pH 5.5 to 6.5 and sodium chloride is added to make it isotonic. The solution obtained is filtered free from pyrogens and the filtrate is transferred under aseptic conditions into ampoules which are then sterilised and sealed by fusion.

### F) Suppositories

| | | |
|---|---|---|
| flibanserin hydrochloride | 50 | mg |
| solid fat | 1650 | mg |
| | 1700 | mg |

The hard fat is melted. At 40° C. the ground active substance is homogeneously dispersed. It is cooled to 38° C. and poured into slightly chilled suppository moulds.

In a particular preferred embodiment of the instant invention, flibanserin is administered in form of specific film coated tablets. Examples of these preferred formulations are listed below. The film coated tablets listed below can be manufactured according to procedures known in the art (see hereto WO 03/097058).

### G) Film Coated Tablet

### Core

| Constituents | mg/tablet |
|---|---|
| Flibanserin | 25.000 |
| Lactose monohydrate | 71.720 |
| Microcrystalline cellulose | 23.905 |
| HPMC (Methocel E5) | 1.250 |
| Carboxymethylcellulose sodium | 2.500 |
| Magnesium stearate | 0.625 |

### Coating

| Constituents | mg/tablet |
|---|---|
| HPMC (Methocel E5) | 1.440 |
| Polyethylene Glycol 6000 | 0.420 |
| Titanium dioxide | 0.600 |
| Talc | 0.514 |
| Iron oxide red | 0.026 |
| Total Film coated tablet | 128.000 |

### H) Film Coated Tablet

### Core

| Constituents | mg/tablet |
|---|---|
| Flibanserin | 50.000 |
| Lactose monohydrate | 143.440 |
| Microcrystalline cellulose | 47.810 |
| HPMC (e.g. Pharmacoat 606) | 2.500 |
| Carboxymethylcellulose sodium | 5.000 |
| Magnesium stearate | 1.250 |

### Coating

| Constituents | mg/tablet |
|---|---|
| HPMC (e.g. Pharmacoat 606) | 2.400 |
| Polyethylene Glycol 6000 | 0.700 |
| Titanium dioxide | 1.000 |
| Talc | 0.857 |
| Iron oxide red | 0.043 |
| Total Film coated tablet | 255.000 |

### I) Film Coated Tablet

### Core

| Constituents | mg/tablet |
|---|---|
| Flibanserin | 100.000 |
| Lactose monohydrate | 171.080 |
| Microcrystalline cellulose | 57.020 |
| HPMC (e.g. Methocel E5) | 3.400 |
| Carboxymethylcellulose sodium | 6.800 |
| Magnesium stearate | 1.700 |

### Coating

| Constituents | mg/tablet |
|---|---|
| HPMC (e.g. Methocel E5) | 3.360 |
| Polyethylene Glycol 6000 | 0.980 |
| Titanium dioxide | 1.400 |
| Talc | 1.200 |
| Iron oxide red | 0.060 |
| Total Film coated tablet | 347.000 |

### J) Film Coated Tablet

### Core

| Constituents | mg/tablet |
|---|---|
| Flibanserin | 2.000 |
| Dibasic Calciumphosphate, anhydrous | 61.010 |
| Microcrystalline cellulose | 61.010 |
| HPMC (Methocel E5) | 1.950 |
| Carboxymethylcellulose sodium | 2.600 |
| Colloidal silicon dioxide | 0.650 |
| Magnesium stearate | 0.780 |

### Coating

| Constituents | mg/tablet |
|---|---|
| HPMC (Methocel E5) | 1.440 |
| Polyethylene Glycol 6000 | 0.420 |
| Titanium dioxide | 0.600 |
| Talc | 0.514 |
| Iron oxide red | 0.026 |
| Total Film coated tablet | 133.000 |

### K) Film Coated Tablet

### Core

| Constituents | mg/tablet |
|---|---|
| Flibanserin | 100.000 |
| Dibasic Calciumphosphate, anhydrous | 69.750 |
| Microcrystalline cellulose | 69.750 |
| HPMC (e.g. Methocel E5) | 2.750 |
| Carboxymethylcellulose sodium | 5.000 |
| Colloidal silicon dioxide | 1.250 |
| Magnesium stearate | 1.500 |

### Coating

| Constituents | mg/tablet |
|---|---|
| HPMC (e.g. Methocel E5) | 2.400 |
| Polyethylene Glycol 6000 | 0.700 |
| Titanium dioxide | 1.043 |
| Talc | 0.857 |
| Total Film coated tablet | 255.000 |

### L) Film Coated Tablet

### Core

| Constituents | mg/tablet |
|---|---|
| Flibanserin | 20.000 |
| Lactose monohydrate | 130.000 |
| Microcrystalline cellulose | 43.100 |
| Hydroxypropyl Cellulose (e.g. Klucel LF) | 1.900 |
| Sodium Starch Glycolate | 4.000 |
| Magnesium stearate | 1.000 |

### Coating

| Constituents | mg/tablet |
|---|---|
| HPMC (e.g. Methocel E5) | 2.400 |
| Polyethylene Glycol 6000 | 0.700 |
| Titanium dioxide | 1.043 |
| Talc | 0.857 |
| Total Film coated tablet | 205.000 |

What is claimed is:

1. A method for the treatment of Attention Deficit Hyperactivity Disorder comprising administering a therapeutically effective amount of flibanserin, or a pharmacologically acceptable acid addition salt thereof.

2. The method as recited in claim 1, wherein the pharmaceutically acceptable acid addition salt of flibanserin is selected from a salt formed by acids selected from succinic acid, hydrobromic acid, acetic acid, fumaric acid, maleic acid, methanesulphonic acid, lactic acid, phosphoric acid, hydrochloric acid, sulphuric acid, tartaric acid, citric acid, and mixtures thereof.

3. The method as recited in claim 1, wherein the flibanserin is administered in a dosage range of between about 0.1 to about 400 mg per day.
